(19) United States
(12) Patent Application Publication
(10) Pub. No.: US 2025/0221850 A1
(43) Pub. Date: Jul. 10, 2025

(54) Method and System For Evaluating Performance of Phacoemulsification Systems

(71) Applicant: JOHNSON & JOHNSON SURGICAL VISION, INC., Irvine, CA (US)

(72) Inventors: Assaf Govari, Haifa (IL); Vadim Gliner, Haifa (IL)

(21) Appl. No.: 18/409,420

(22) Filed: Jan. 10, 2024

(51) Int. Cl. A61F 9/007; A61B 17/00; A61B 90/00; G16H 50/30

(52) U.S. Cl. CPC A61F 9/00709; G16H 50/30; A61B 2017/00022; A61B 2090/064

(57) ABSTRACT

A method, ophthalmic surgical system, and a computer program product for assessing and providing a clinical index during an ophthalmic procedure performed on a patient, the method including: providing an ophthalmic surgical system and at least one sensor coupled with the ophthalmic surgical system for sensing a parameter within an eye of the patient; obtaining a first sequence of measured values, the first sequence of values indicating measurements taken by the at least one sensor at a plurality of points in time during the medical procedure; assessing the clinical index based at least on a function of the first sequence of measured values, the function involving a deviation of each value of the first sequence of measured values from a target; and providing to a user of the medical system an indication of the clinical index.

### FIELD OF THE DISCLOSURE

This disclosure relates generally to a method for determining and relaying information related to the performance of an ophthalmologic operation, and more specifically to a method and apparatus for providing a clinical index of an operation to evaluate the procedure, including the risk to the surgical site due to the operation.

### BACKGROUND OF THE DISCLOSURE

Ophthalmic surgery poses significant challenges both to the developers of the equipment and to its users, such as physicians.

One significant challenge relates to maintaining the intraocular pressure (IOP) within an eye of the patient operated upon, for example within a predetermined range, such as 20-200 mmHg.

A particular operation type is a cataract removal operation. A cataract is a clouding and hardening of the eye's natural lens, which often happens when people get older. A common treatment of cataract is phacoemulsification cataract surgery. In the procedure, a portion of the anterior surface of the lens capsule is removed to gain access to the cataract. The surgeon then uses a phacoemulsification probe, which is an ultrasonic handpiece with a needle and a sleeve. The tip of the needle vibrates at ultrasonic frequency, which emulsifies the cataract lens. At a same time, a pump aspirates particles and fluid from the eye through the tip, wherein the aspirated fluids are replaced with irrigation of a balanced salt solution to maintain the intraocular pressure in the anterior chamber of the eye. After removing the cataract with phacoemulsification, the softer outer lens cortex is removed with suction. An intraocular lens is then introduced into the empty lens capsule restoring the patient's vision.

Due to the combination of irrigation and aspiration involved in the procedure, maintaining the IOP in cataract operations is particularly important but nonetheless challenging. Therefore, there is a need for a method and system for providing a clinical index that represents the effect of the IOP and possibly additional factors have on the patient's eye, in phacoemulsification and also in other operations.

### BRIEF DESCRIPTION OF THE DRAWINGS

The present disclosure will be more fully understood from the following detailed description of the examples thereof, taken together with the drawings, in which:

FIG. 1A is a schematic, pictorial view of a phacoemulsification apparatus, in accordance with an example of the present disclosure;

FIG. 2A is an exemplary schematic illustration of a display showing the clinical index of an operation, in accordance with some examples of the disclosure;

FIG. 2B is an exemplary schematic illustration of a display showing the clinical index of an operation, in accordance with some examples of the disclosure;

FIG. 3 is a flowchart of steps in a method for determining and providing a clinical index to a user of an ophthalmic surgical system, in accordance with some exemplary examples of the disclosure; and

FIG. 4 is a block diagram of a computing platform for determining and providing a clinical index to a user of an ophthalmic surgical system, in accordance with some exemplary examples of the disclosure.

### DETAILED DESCRIPTION OF EXAMPLES

In the following description, numerous specific details are set forth in order to provide a thorough understanding of the present invention. It will be apparent to one skilled in the art, however, that the present invention may be practiced without these specific details. In other instances, well-known circuits, control logic, and the details of computer program instructions for conventional algorithms and processes have not been shown in detail in order not to obscure the present invention unnecessarily.

Software programming code, which embodies aspects of the present invention, is typically maintained in permanent storage, such as a computer readable medium. In a client-server environment, such software programming code may be stored on a client or a server. The software programming code may be embodied on any of a variety of known media for use with a data processing system. This includes, but is not limited to, magnetic and optical storage devices such as disk drives, magnetic tape, compact discs (CD's), digital video discs (DVD's), and computer instruction signals embodied in a transmission medium with or without a carrier wave upon which the signals are modulated. For example, the transmission medium may include a communications network, such as the Internet. In addition, while the invention may be embodied in computer software, the functions necessary to implement the invention may alternatively be embodied in part or in whole using hardware components such as application-specific integrated circuits or other hardware, or some combination of hardware components and software.

In the description below, the term “about” as related to numerical values may include values that are in the range of +/−10% of the indicated value.

### Overview

A cataract is often treated by a phacoemulsification operation, in which a needle of an ultrasonic handpiece is inserted into a patient's eye, wherein the needle can vibrate at ultrasonic frequency, to emulsify the cataract.

During the operation, a pump aspirates particles and fluid from the eye through the tip. An irrigation system irrigates a balanced salt solution to compensate for the aspirated material and maintain the IOP. The aspiration and irrigation may be coordinated such that the IOP in the anterior chamber of the eye is maintained within a predetermined range and as stable as possible.

The well-being of the eye during and after the operation may be affected by a plurality of factors. Some of the factors may depend on the performance of the equipment used during the procedure, for example the effectivity of the equipment in continuously maintaining the desired IOP, while other factors may be subject to the expertise of the user performing the operation, such as the physician. One or more of the factors detailed below, and optionally additional factors may be used for calculating a clinical index which may indicate a risk to the eye due to the operation.

The clinical index may be accumulated during the whole operation, and may provide an important measure for assessing the overall possible damage that was possibly caused to the eye due to the operation.

One such factor is the intraocular pressure (IOP) within the eye. A target_IOP value may be defined as a default, or set for a specific patient. While it is desired to maintain the IOP at the target value as much as possible, it is appreciated that the IOP may deviate from the target value. However, it is desired that such deviations are minimized. Thus, this aspect of the operation quality may be represented by a value calculated by integrating the absolute value of the deviation of the IOP from the target value over the operation, or a linear function of this absolute value.

In some examples, the possible damage to the eye that is caused by deviations of the IOP from the target value may vary non-linearly in accordance with the extent of the deviation. For example, one significant deviation from the target_IOP can cause a more severe and irreversible damage than a plurality of small deviations which are kept within a safe range and may be harmless. Thus, it is desired that larger deviations will yield a significantly higher clinical index. Thus, this aspect of the clinical index may be calculated by integrating over a non-linear function of the absolute value of the deviation of the IOP from the target value over the operation. For example, a value of an exponential function of the absolute value may be integrated over time.

Another such factor may be the amount of balanced salt solution that is irrigated into the eye (amount used), to replace the aspirated fluids. Since irrigating the eye with fluid can cause endothelial cell loss, it is also desired to minimize the amount of irrigated fluid used. Thus, this aspect of the operation quality may be represented as a value calculated by integrating the amount of balanced salt solution irrigated into the eye during the operation, or a function thereof.

Yet another such factor may be the ultrasonic energy emitted during the operation by the ultrasonic handpiece used for emulsifying the cataract. It is appreciated that it is desired to minimize the amount of energy emitted into the eye to avoid/minimize damage caused by the heat generated. Thus, this aspect of the operation quality may be represented as a value calculated by integrating the amount of the emitted ultrasonic energy, or a function thereof.

In some examples, one or more of the factors above, or additional factors, may be combined to provide a clinical index for the operation. For example, the clinical index of the operation may be calculated as a linear combination of: an integral over the operation duration of a function of the absolute value of the deviation of the IOP from the target value, and: an integral over the operation duration of a function of the amount of balanced salt solution irrigated into the eye, and/or an integral over the operation duration of a function of the amount of the emitted ultrasonic energy. It is appreciated that any of the functions may be the identity function, a linear function, an exponential function, or the like.

The clinical index may be presented to the user at the end of the operation in any required form, comprising for example text, graphs, or the like. It will be appreciated that since the values are integrated during the operation, the index is constantly non-decreasing.

In some examples, the values may be presented as text comprising the clinical index value.

In some examples, a graph may show the clinical index as integrated over the entire procedure. It is appreciated that the graph presenting the clinical index over the entire operation is non-decreasing.

In some examples, the values may be presented in comparison to a reference value. For example, the reference value may be collected from a plurality of operations performed by one or more users, such as expert or experienced physicians.

In some examples, when the clinical index is accumulated over time, its graph may be scaled to match the average length of the operations upon which the reference clinical index is calculated.

In some examples, if the values are combined from multiple factors, a user can view separately one or more of the factors. For example, the user can view a graph of the function of the IOP (or any of the other factors) as integrated.

### System Description

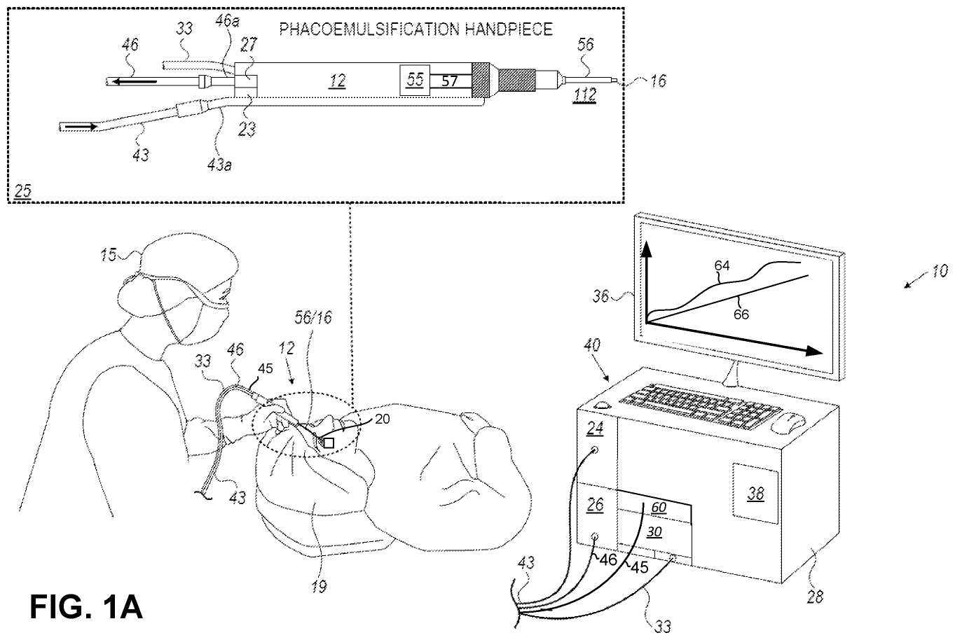

Referring now to FIG. 1A, showing an exemplary phacoemulsification apparatus 10, which includes a phacoemulsification probe 12 having at its distal end 112 a needle 16 which may comprise an optical fiber therein. Needle 16 is configured to be inserted by a physician 15 into the lens capsule of an eye 20 of a patient 19 to remove a cataract lens. While needle 16 is shown in inset 25 as a straight object, it is appreciated that any suitable needle may be used with phacoemulsification probe 12, for example, a curved or bent tip needle commercially available from Johnson & Johnson Surgical Vision, Inc., Irvine, CA, USA.

System 10 may comprise console 28, comprising a user interface 40, including physical and virtual controls such as a keyboard, a mouse, a touchscreen, a joystick, a foot pedal, a speaker, a microphone, or others, for inputting data or commands to the apparatus or receiving data from the apparatus, and a processor 38.

In an example, system 10 may comprise a display 36 for displaying to the physician various images or textual information related to the operation or to the patient, including aspects of the operation, for example, graph 64 of the clinical index as accumulated during the operation, and a reference graph 66. Further display options are exemplified below.

In some examples, one or more controls of user interface 40 and display 36 may be integrated into a touch screen graphical user interface.

Probe 12 may comprise ultrasound transducer 55, e.g., a piezoelectric ultrasound transducer, which is configured to vibrate horn 57 and needle 16 in one or more resonant vibration modes of the combined horn and needle element. During the phacoemulsification operation, upon the application of one or more drive signals to ultrasound transducer 55, the vibration of needle 16 is used to emulsify the cataract. Ultrasound transducer 55 and horn 57, or different combinations providing the same effect are collectively referred to as an ultrasound transducer. Ultrasound transducer 55 may be toggled ON/OFF by controller 60 through cable 45, in accordance with whether needle 16 is in contact with the eye or not, or in accordance with the physician activation.

In some examples, eye fluid and waste matter (e.g., emulsified parts of the cataract) are aspirated via a lumen in needle 16 to a collection receptacle (not shown) by an aspiration pump 26, which may be controlled by processor 38, using aspiration tubing line 46 running from aspiration channel 46a of probe 12 to console 28.

In some examples, probe 12 may further comprise a coaxial irrigation sleeve 56 that at least partially surrounds needle 16. During the phacoemulsification operation, an irrigation pump 24 which may be controlled by processor 38 may pump irrigation fluid from an irrigation reservoir (not shown) to irrigation sleeve 56, to irrigate the eye. The fluid may be pumped via an irrigation tubing line 43 running from console 28 to an irrigation channel 43a of probe 12.

In some examples, irrigation pump 24 and aspiration pump 26 may be controlled by processor 38 in accordance with readings received from irrigation sensor 23 and aspiration sensor 27, respectively, to maintain the IOP within predetermined limits. An overall pressure within the eye chamber may be assessed, for example by processor 38, based on the readings received from irrigation sensor 23 and aspiration sensor 27.

In further examples, the IOP may be sensed by a sensor positioned within the eye, in the handpiece 12, in the console 28 and/or anywhere along irrigation tubing line 43 and/or aspiration line 46 and connectable to console 28 or to a processing unit.

Processor 38 is thus adapted to control the operation of various functions of probe 12 such as the irrigation and aspiration, in accordance with the physician's commands as provided via user interface 40, and with various measurements.

In some examples, processor 38 may automatically control the irrigation and aspiration in order to maintain a required pressure level within the chamber. Processor 38 may also be configured for measuring or otherwise determining in an ongoing manner the amount of fluid irrigated into the eye.

It is appreciated that some or all of the functions of processor 38 may be combined in a single physical component. Alternatively, processor 38 may be implemented using multiple physical components. These physical components may comprise hard-wired or programmable devices, or a combination thereof. In some examples, at least some of the functions of processor 38 may be carried out by suitable software stored in a memory device of processor 38 or console 28. This software may be downloaded to a device in electronic form, over a network, or the like. Alternatively, or additionally, the software may be stored in tangible, non-transitory computer-readable storage media, such as optical, magnetic, or electronic memory.

The system may comprise drive module 30 for activating ultrasound transducer 55, for example setting the operation parameters and supplying current to ultrasound transducer 55 using for example electrical wiring running within cable 33. Drive module 30 may be realized in hardware or software, for example, in a proportional-integral-derivative (PID) control architecture.

It is understood that in some examples, the lines between the console and the handpiece, which are required for driving and/or controlling the handpiece, the fluidics, and/or the AVS, such as the irrigation line, aspiration line, a line for controlling the ultrasound transducer, and a line for controlling the AVS, may be provided within a single cable or a plurality of cables, depending on ergonomic, safety, maintenance complexity, and cost considerations.

Drive module 30 may also be configured for determining in an ongoing manner the amount of ultrasonic energy emitted by ultrasound transducer 55.

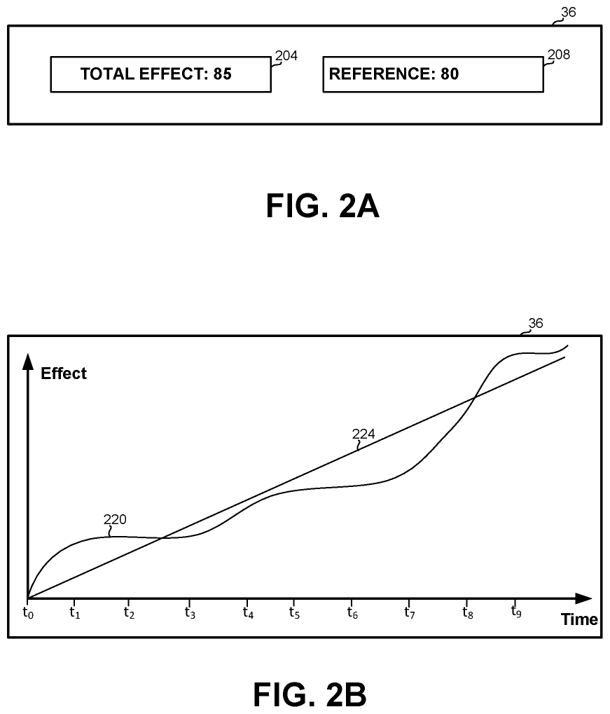

Referring now to FIGS. 2A-2B, illustrating exemplary display options for the clinical index, in accordance with some examples of the disclosure.

FIG. 2A shows a textual display over display device 36, showing index 204 as accumulated since the beginning of the procedure, and a reference index 208, such that the user can compare the clinical index to a reference clinical index which may have been determined upon a plurality of operations of the same type, such as phacoemulsification. It is appreciated that prior to the end of the operation, reference 208 can be shown to relate to the duration of the procedure at the time of the display, in order for accumulated effect 204 and reference 208 to be comparable.

FIG. 2B shows a graphic display, comprising a graph 220 of the index as integrated during the operation, and an accumulated reference graph 224. It is appreciated that the clinical index is accumulative and all values are non-negative (no factor can repair damage caused to the eye), therefore graphs 220 and 224 are non-decreasing. As the operation progresses, the scaling of graphs 220 and 224 may decrease as updated index calculations are added to the graphs on the right hand side.

In some examples, certain areas of the graph of FIG. 2B may be color coded to emphasize their contribution to the clinical index. For example, areas whose contribution to the clinical index is below a predetermined threshold may be colored in green, while areas whose contribution to the clinical index is above a second predetermined threshold may be colored in red.

It is appreciated that further presentation modes may be applied, for example a bar graph, a histogram, or the like.

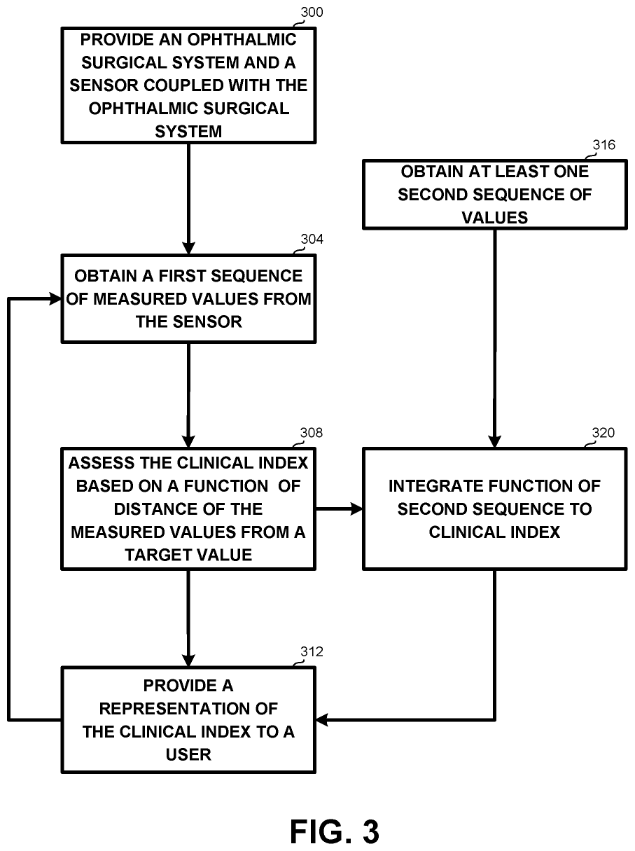

Referring now to FIG. 3, showing a flowchart of a method for determining and providing a clinical index to a user of an ophthalmic surgical system, in accordance with some examples of the disclosure.

At step 300 an ophthalmic surgical system may be provided. The ophthalmic surgical system may comprise a probe, also referred to as a handpiece and a surgical console coupled with the handpiece. A sensor coupled with the ophthalmic surgical system may also be provided, whether as part of the ophthalmic surgical system or as an external unit.

For example, the ophthalmic surgical system may be a phacoemulsification system as shown in FIG. 1A, and the sensor may be an IOP sensor. The IOP sensor may be embedded within the phacoemulsification system, for example as part of an irrigation tube of the system, or may be external to the phacoemulsification system.

At step 304, a first sequence of values may be received from the sensor during an operation. For example, a series of IOP measurements may be received during a phacoemulsification operation. The values may be received at predetermined time intervals. The values may be received directly from a driver of the sensor, from console 28, or the like.

At step 308, a clinical index may be assessed based on a function of the first sequence of values.

In the example above, in order not to harm the patient's eye, the TOP of the patient should be as close as possible to a target value, such as between 20 mmHg and 200 mmHg. Thus, the clinical index may be calculated by integrating the absolute value of the difference between the measured values and the target value, according to the following formula:

$${\int }_{t} \vert IOP ( t ) - target\_IOP \vert dt \qquad ( 1 )$$

wherein IOP(t) is the intraocular pressure as measured at time t,
target_IOP is a predetermined desired value for the IOP, and
the integration is over the phacoemulsification operation or part thereof.

In some examples, under-pressure does not harm the eye, and therefore may not be considered as part of the clinical index computation, such that the formula may be adapted to:

$${\int }_{t} max ( 0 , IOP ( t ) - target\_IOP ) dt \qquad ( 1 a )$$

In some examples, the damage to the eye that may be caused by the IOP being above or below the target value may increase non-linearly with the distance of the measured value from the target value. In this case, the formula may take the form of formula (2) below:

$${\int }_{t} {e}^{\vert IOP ( t ) - target \_ IOP \vert} dt \qquad ( 2 )$$

As above, in some examples only over pressure situations may be considered in the clinical index computation, such that the formula may be:

$${\int }_{t} {e}^{( max ( 0 , IOP ( t ) - target \_ IOP )} dt \qquad ( 2 a )$$

It is appreciated that formulas (1), (1a), (2), and (2a) are exemplary only, and other functions which are linear or non-linear functions of the difference may be used.

In some examples, small deviations from the target_IOP may be ignored, such that a difference between the IOP and the target_IOP within an acceptable range will contribute zero to the clinical index. Thus, formulas (1), (1a), (2), and (2a) above will be applicable only to IOP values that deviate in at least the threshold from the target_IOP.

The acceptable range may be set to a default applicable to all patients, or to a value adapted, for example by a user, to a specific patient.

At step 312, the clinical index may be provided to a user such as a physician operating the ophthalmic surgical system or to another person such as a supervisor of the physician.

The index may be provided by displaying the index value, sending a message, storing a value in a database, or in any other form.

When the critical index is displayed to the user, it may be displayed alongside or in another comparative form to a reference value.

If the clinical index is displayed to a user over a display device, it may be displayed textually or graphically, as demonstrated in FIGS. 2A-2B above.

In some examples, the clinical index may be calculated upon information additional to the measures received from the sensor.

Thus, at step 316, at least one second sequence of values that relate to a factor that should be minimized during the operation may be obtained.

At step 320, a function of the at least one second sequence may also be integrated into the calculation to obtain the clinical index.

In one example, the second sequence may comprise the amount of ultrasonic energy emitted by the ultrasonic phacoemulsification probe.

The values may be integrated, such that the clinical index may be described by the following formula:

$$a {\int }_{t} \vert IOP ( t ) - {target}_{IOP} \vert dt + b {\int }_{t} E ( t ) dt \qquad ( 3 a )$$

$$or$$

$$a {\int }_{t} {e}^{\vert IOP ( t ) - target \_ IOP \vert} dt + b {\int }_{t} E ( t ) dt \qquad ( 3 b )$$

wherein E(t) may represent the amount of ultrasonic energy emitted by the ultrasonic actuator of the phacoemulsification system, and
wherein a and b may be equal or non-equal predetermined numerical weights.

The energy may be estimated, for example, by the electrical power of the ultrasonic actuator, which may be obtained by multiplying the current and the voltage.

In another example, the second sequence may comprise the amount of balanced salt solution irrigated into the eye during the medical procedure such as the phacoemulsification operation, to replace the eye fluid aspirated. The amount may be integrated, such that the clinical index may be described by the following formula:

$$a {\int }_{t} \vert IOP ( t ) - {target}_{IOP} \vert dt + b {\int }_{t} B ( t ) dt \qquad ( 3 c )$$

$$or$$

$$a {\int }_{t} {e}^{\vert IOP ( t ) - target \_ IOP \vert} dt + b {\int }_{t} B ( t ) dt \qquad ( 3 d )$$

wherein B(t) may represent the amount of balanced salt solution irrigated into the eye at time t.

The solution amount may be estimated, for example, by obtaining the number of pump cycles used for pumping the solution (e.g., using an encoder coupled with the pump) and as reported by a controller of the pump, multiplied by the amount of solution pumped on every cycle.

In some examples, more than one factor may be used for calculating the clinical factor in addition to the IOP, for example the amount of ultrasonic energy emitted by the ultrasonic actuator of the phacoemulsification system, and the amount of balanced salt solution irrigated into the eye during the phacoemulsification operation. In such examples, the clinical index may be described by the following formula:

$$a {\int }_{t} \vert IOP ( t ) - {target}_{IOP} \vert dt + b {\int }_{t} E ( t ) dt + c {\int }_{t} B ( t ) dt \qquad ( 3 e )$$

$$or$$

$$a {\int }_{t} {e}^{\vert IOP ( t ) - target \_ IOP \vert} dt + b {\int }_{t} E ( t ) dt + c {\int }_{t} B ( t ) dt \qquad ( 3 f )$$

wherein c is also a predetermined numerical weight, which may or may not be equal to a or b.

It is appreciated that the clinical index according to any of the formulas above may be calculated such that the integrations are over the whole duration of the phacoemulsification operation. However, in some examples, the clinical index may be calculated dynamically and accumulatively, such that at every point in time during the operation, the clinical index as integrated until that point is available and may be presented to a user.

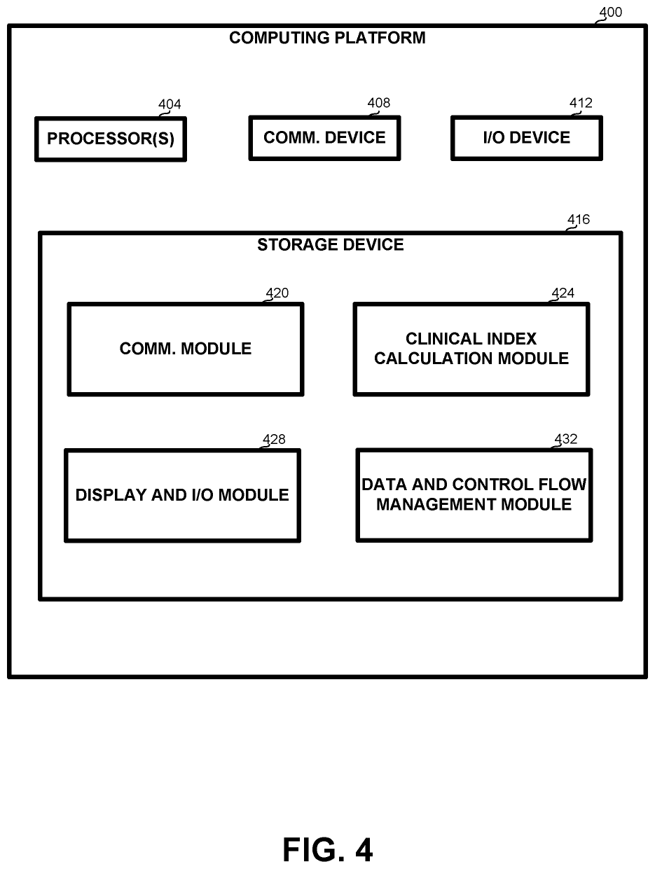

Referring now to FIG. 4, showing a block diagram of a computing platform 400 for determining and displaying a clinical index, in accordance with some examples of the disclosure.

It is appreciated that computing platform 400 may be embedded within console 28, but may also be a standalone computing platform or embedded elsewhere and be in operative communication with console 28.

Computing platform 400 may be implemented as one or more computing platforms which may be operatively connected to each other. For example, one or more remote computing platforms, which may be implemented for example on a cloud computer. Other computing platforms may be a part of a computer network of the associated organization. In other examples, all the functionalities may be provided by one or more computing platforms all being a part of the organization network.

Computing platform 400 may comprise one or more processors 404 located on the same computing platform or not, which may be one or more Central Processing Units (CPU), microprocessors, electronic circuits, Integrated Circuits (IC) or the like. Processor 404 may be configured to provide the required functionality, for example by loading to memory and activating the software modules stored on storage device 416 detailed below.

Computing platform 400 may comprise a communication device 408 for communicating with other components of the phacoemulsification system such as one or more sensors, other sensors or devices, or other computing platforms as necessary, for example obtaining readings from one or more pressure sensors, obtaining readings related to the energy emitted by the ultrasonic vibrator or to the volume of balanced salt solution used, storing data on remote storage devices, or the like. Communication module 408 may be adapted to interface with any communication channel such as Local Area Network (LAN), Wide Area Network (WAN), cellular network or the like, and use any relevant communication protocol.

Computing platform 400 may comprise an Input/Output (I/O) device 412, such as a display device, for displaying information such as clinical index values, comparative information, or the like. I/O device 412 may also be operative in receiving instructions from a user, for example changing the display settings. In some examples, I/O device 412 may be display device 36 of FIG. 1A.

Computing platform 400 may comprise a storage device 416, such as a hard disk drive, a Flash disk, a Random Access Memory (RAM), a memory chip, or the like. In some exemplary examples, storage device 416 may retain program code operative to cause processor 404 to perform acts associated with any of the modules listed below, or steps of the method of FIG. 3 above. The program code may comprise one or more executable units, such as functions, libraries, standalone programs or the like, adapted to execute instructions as detailed below.

Alternatively, or additionally, the provided instructions may be stored on non-transitory tangible computer-readable media, such as magnetic, optical, or electronic memory.

Storage device 416 may comprise communication module 420 for transmitting and receiving data to and from other components of the system or other systems through communication device 408, for example receiving pressure readings, readings or data from other sensors or controllers or drivers, user settings, or the like, and/or sending clinical index values, display settings, or the like.

Storage device 416 may comprise clinical index calculation module 424 for determining the clinical index based on IOP readings, and optionally one or more additional factors such as amount of balanced salt solution used, emitted ultrasonic energy, or the like.

Storage device 416 may comprise display and I/O module 428, for rendering a display to the user on display device 36, such as textual values of clinical index as shown in FIG. 2A, graphs of the clinical index as shown in FIG. 2B, or the like. Display and I/O module 428 may also be operative in receiving instructions and settings from the user.

Storage device 416 may comprise data and control flow management module 432, for activating the modules above in the correct order and timing, and with the required input, for example determining the clinical index based on the received readings, determining, or updating the graph showing the clinical index, or the like.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembly instructions, instruction-set-architecture instructions, machine instructions, machine dependent instructions, microcode, firmware instructions, state-setting data, or either source code or object code written in any combination of one or more programming languages, programming languages such as Java, C, C++, Python, or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

A method for assessing and providing a clinical index during an ophthalmic procedure performed on a patient, comprising: providing an ophthalmic surgical system and at least one sensor coupled with the ophthalmic surgical system for sensing a parameter within an eye of the patient; obtaining a first sequence of measured values, the first sequence of values indicating measurements taken by the at least one sensor at a plurality of points in time during the medical procedure; assessing the clinical index based at least on a function of the first sequence of measured values, the function involving a deviation of each value of the first sequence of measured values from a target; and providing to a user of the medical system an indication of the clinical index.

### Example 2

The method according to example 1, wherein each measured value from the first sequence of measured values indicates an intraocular pressure (IOP) within an eye of the patient at a point in time during the medical procedure.

### Example 3

The method according to any of examples 1-2, wherein the function includes an integral over a period of time at which the medical procedure was taking place, of a second function of an absolute value of a deviation of the IOP from the predetermined threshold.

### Example 4

The method according to any of examples 1-3, wherein the second function is the identity function.

### Example 5

The method according to any of examples 1-4, wherein the second function increases faster than a linear function or the second function is an exponential function.

### Example 6

The method according to any of examples 1-5, further comprising obtaining at least one second sequence of values, and wherein the function is also based on the second sequence of values.

### Example 7

The method according to any of examples 1-6, wherein the second sequence of values indicates energy emitted by an ultrasonic transducer at a second plurality of points in time during the medical procedure, and wherein the function includes an integral over of a third function of the energy during a period of time at which the medical procedure was taking place.

### Example 8

The method according to any of examples 1-7, wherein the second sequence of values indicates a flow of balanced salt solution irrigated into an eye of the patient at a second plurality of points in time during the medical procedure.

### Example 9

The method according to any of examples 1-8 wherein the function includes an integral over of a third function of the flow over a period of time at which the medical procedure was taking place.

### Example 10

A method for evaluating a clinical index during a phacoemulsification operation performed on a patient, comprising: providing a phacoemulsification system and at least one sensor coupled with the phacoemulsification system, for sensing an IOP within an eye of the patient; obtaining a first sequence of values from the at least one sensor, the first sequence of values indicating an IOP measure at a first plurality of points in time during the phacoemulsification operation; obtaining a second sequence of values, each value from the second sequence of values indicating energy emitted by an ultrasonic transducer at a second plurality of points in time during the phacoemulsification operation; obtaining a third sequence of values, each value from the third sequence of values indicating a flow of balanced salt solution (B) irrigated into an eye of the patient at a third plurality of points in time during the phacoemulsification operation; assessing the clinical index based on a following formula:

$$a {\int }_{t} {e}^{\vert IOP ( t ) - target \_ IOP \vert} dt + b {\int }_{t} E ( t ) dt + c {\int }_{t} B ( t ) dt$$

$$or$$

$$a {\int }_{t} {e}^{max ( 0 , IOP ( t ) - target \_ IOP )} dt + b {\int }_{t} E ( t ) dt + c {\int }_{t} B ( t ) dt$$

wherein a, b, and c are predetermined numerical weights, wherein c may or may not be equal to either a or b, t is time from the beginning of the medical procedure, IOP(t) is a value of the first sequence at time t, target_IOP is an IOP value desired to be maintained during the medical procedure, E(t) is value of the second sequence at time t, and B(t) is value of the third sequence at time t; and providing to a user of the phacoemulsification system an indication of the clinical index.

### Example 11

An ophthalmic surgical system, comprising: a phacoemulsification probe for performing a phacoemulsification operation, the phacoemulsification probe having a needle at its distal end, the needle configured to be inserted into an eye of a patient; a pressure sensor; and a processor, configured to repeatedly: obtain a first sequence of measured values, the first sequence of values indicating a measurement taken by the at least one sensor during the phacoemulsification operation; assess the clinical index based at least on a function of the sequence of measured values, the function involving a deviation from a threshold; and provide to a user of the medical system an indication of the clinical index to the patient.

### Example 12

The ophthalmic surgical system according to example 11, wherein each measured value from the first sequence of measured values indicates an IOP within an eye of the patient at a point in time during the phacoemulsification operation.

### Example 13

The ophthalmic surgical system according to any of examples 11-12, wherein the function includes an integral over a period of time at which the medical procedure was taking place, of a second function of an absolute value of a deviation of the IOP from the predetermined threshold, wherein the second function increases faster than a linear function.

### Example 14

The ophthalmic surgical system according to any of examples 11-13 wherein the second function is an exponential function.

### Example 15

The ophthalmic surgical system according to any of claims 11-14 wherein the processor is further configured to obtain at least one second sequence of values, and wherein the function is also based on the second sequence of values.

### Example 16

The ophthalmic surgical system according to any of examples 11-15, wherein the second sequence of values indicates energy emitted by an ultrasonic transducer at a second plurality of points in time during the phacoemulsification operation.

### Example 17

The ophthalmic surgical system according to any of examples 11-16, wherein the second sequence of values indicates a flow of balanced salt solution irrigated into an eye of the patient at a second plurality of points in time during the phacoemulsification operation.

### Example 18

The ophthalmic surgical system according to any of examples 11-17, wherein the function includes an integral over a period of time at which the phacoemulsification operation was taking place, of the flow.

### Example 19

The ophthalmic surgical system according to any of examples 11-18, wherein each measured value from the first sequence of measured values indicates an IOP within an eye of the patient at a point in time during the phacoemulsification operation, and wherein the processor is further configured to: obtain a second sequence of values, the second sequence of values indicating energy emitted by an ultrasonic transducer at a second plurality of points in time during the phacoemulsification operation; obtain a third sequence of values, each value from the third sequence of values indicating a flow of balanced salt solution (B) irrigated into an eye of the patient at a third plurality of points in time during the phacoemulsification operation; assessing the clinical index based on a following formula:

$$a {\int }_{t} {e}^{\vert IOP ( t ) - target \_ IOP )} dt + b {\int }_{t} E ( t ) dt + c {\int }_{t} B ( t ) dt$$

$$or$$

$$a {\int }_{t} {e}^{max ( 0 , IOP ( t ) - target \_ IOP )} dt + b {\int }_{t} E ( t ) dt + c {\int }_{t} B ( t ) dt$$

wherein t is time from the beginning of the phacoemulsification operation, IOP(t) is a value of the first sequence at time t, target(IOP) is an IOP value desired to be maintained during the phacoemulsification operation, E(t) is value of the second sequence at time t, B(t) is value of the third sequence at time t, and a, b, and c are predetermined numerical weights; and providing to a user of the phacoemulsification system an indication of the clinical index.

### Example 20

A computer program product comprising a non-transitory computer readable medium retaining program instructions, which instructions when read by a processor, cause the processor to perform: providing a medical system and at least one sensor coupled with the medical system for sensing a parameter within a body of the patient; obtaining a first sequence of measured values, the first sequence of values indicating measurements taken by the at least one sensor at a plurality of points in time during the medical procedure; assessing the clinical index based at least on a function of the first sequence of measured values, the function involving a deviation of each value of the first sequence of measured values from a threshold; and providing to a user of the medical system an indication of the clinical index.

### Example 21

The method according to any of examples 1-9, wherein said assessing and said providing to the user are performed dynamically during the ophthalmic procedure.

### Example 22

The method according to any of examples 1-9, wherein said assessing and said providing to the user are performed when the ophthalmic procedure is completed.

### Example 23

The method according to example 10, wherein said assessing and said providing to the user are performed dynamically during the ophthalmic procedure.

### Example 24

The method according to example 10, wherein said assessing and said providing to the user are performed when the ophthalmic procedure is completed.

### Example 25

The ophthalmic surgical system according to any of examples 11-19, wherein said assessing and said providing to the user are performed dynamically during the ophthalmic procedure.

### Example 26

The ophthalmic surgical system according to any of examples 11-19, wherein said assessing and said providing to the user are performed when the ophthalmic procedure is completed.

### Example 27

The ophthalmic surgical system according to any of examples 11-19, wherein the pressure sensor is coupled with an irrigation line of the phacoemulsification system.

### Example 28

The ophthalmic surgical system according to any of examples 11-19, wherein the pressure sensor is external to the phacoemulsification system.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A method for assessing and providing a clinical index during an ophthalmic procedure performed on a patient, comprising:
providing an ophthalmic surgical system and at least one sensor coupled with the ophthalmic surgical system for sensing a parameter within an eye of the patient;
obtaining a first sequence of measured values, the first sequence of values indicating measurements taken by the at least one sensor at a plurality of points in time during the medical procedure;
assessing the clinical index based at least on a function of the first sequence of measured values, the function involving a deviation of each value of the first sequence of measured values from a target; and
providing to a user of the medical system an indication of the clinical index.

2. The method of claim 1, wherein each measured value from the first sequence of measured values indicates an intraocular pressure (IOP) within an eye of the patient at a point in time during the medical procedure.

3. The method of claim 2, wherein the function includes an integral over a period of time at which the medical procedure was taking place, of a second function of an absolute value of a deviation of the IOP from the predetermined threshold.

4. The method of claim 3, wherein the second function is the identity function.

5. The method of claim 3, wherein the second function increases faster than a linear function or the second function is an exponential function.

6. The method of claim 1, further comprising obtaining at least one second sequence of values, and wherein the function is also based on the second sequence of values.

7. The method of claim 6, wherein the second sequence of values indicates energy emitted by an ultrasonic transducer at a second plurality of points in time during the medical procedure, and wherein the function includes an integral over of a third function of the energy during a period of time at which the medical procedure was taking place.

8. The method of claim 6, wherein the second sequence of values indicates a flow of balanced salt solution irrigated into an eye of the patient at a second plurality of points in time during the medical procedure.

9. The method of claim 8, wherein the function includes an integral over of a third function of the flow over a period of time at which the medical procedure was taking place.

10. A method for evaluating a clinical index during a phacoemulsification operation performed on a patient, comprising:
providing a phacoemulsification system and at least one sensor coupled with the phacoemulsification system, for sensing an IOP within an eye of the patient;
obtaining a first sequence of values from the at least one sensor, the first sequence of values indicating an IOP measure at a first plurality of points in time during the phacoemulsification operation;
obtaining a second sequence of values, each value from the second sequence of values indicating energy emitted by an ultrasonic transducer at a second plurality of points in time during the phacoemulsification operation;
obtaining a third sequence of values, each value from the third sequence of values indicating a flow of balanced salt solution (B) irrigated into an eye of the patient at a third plurality of points in time during the phacoemulsification operation;
assessing the clinical index based on a following formula:
$$a {\int }_{t} {e}^{\vert IOP ( t ) - target \_ IOP \vert} dt + b {\int }_{t} E ( t ) dt + c {\int }_{t} B ( t ) dt$$
$$or$$
$$a {\int }_{t} {e}^{max ( 0 , IOP ( t ) - target \_ IOP )} dt + b {\int }_{t} E ( t ) dt + c {\int }_{t} B ( t ) dt$$
wherein
a, b, and c are predetermined numerical weights, wherein c may or may not be equal to either a or b,
t is time from the beginning of the medical procedure,
IOP(t) is a value of the first sequence at time t,
target_IOP is an IOP value desired to be maintained during the medical procedure,
E(t) is value of the second sequence at time t, and
B(t) is value of the third sequence at time t, and
providing to a user of the phacoemulsification system an indication of the clinical index.

11. An ophthalmic surgical system, comprising:
a phacoemulsification probe for performing a phacoemulsification operation, the phacoemulsification probe having a needle at its distal end, the needle configured to be inserted into an eye of a patient;
a pressure sensor; and
a processor, configured to repeatedly:
obtain a first sequence of measured values, the first sequence of values indicating a measurement taken by the at least one sensor during the phacoemulsification operation;
assess the clinical index based at least on a function of the sequence of measured values, the function involving a deviation from a threshold; and
provide to a user of the medical system an indication of the clinical index to the patient.

12. The ophthalmic surgical system of claim 11, wherein each measured value from the first sequence of measured values indicates an IOP within an eye of the patient at a point in time during the phacoemulsification operation.

13. The ophthalmic surgical system of claim 11, wherein the function includes an integral over a period of time at which the medical procedure was taking place, of a second function of an absolute value of a deviation of the IOP from the predetermined threshold, wherein the second function increases faster than a linear function.

14. The ophthalmic surgical system of claim 13, wherein the second function is an exponential function.

15. The ophthalmic surgical system of claim 11, wherein the processor is further configured to obtain at least one second sequence of values, and wherein the function is also based on the second sequence of values.

16. The ophthalmic surgical system of claim 15, wherein the second sequence of values indicates energy emitted by an ultrasonic transducer at a second plurality of points in time during the phacoemulsification operation.

17. The ophthalmic surgical system of claim 15, wherein the second sequence of values indicates a flow of balanced salt solution irrigated into an eye of the patient at a second plurality of points in time during the phacoemulsification operation.

18. The ophthalmic surgical system of claim 17, wherein the function includes an integral over a period of time at which the phacoemulsification operation was taking place, of the flow.

19. The ophthalmic surgical system of claim 11, wherein each measured value from the first sequence of measured values indicates an IOP within an eye of the patient at a point in time during the phacoemulsification operation, and wherein the processor is further configured to:
obtain a second sequence of values, the second sequence of values indicating energy emitted by an ultrasonic transducer at a second plurality of points in time during the phacoemulsification operation;
obtain a third sequence of values, each value from the third sequence of values indicating a flow of balanced salt solution (B) irrigated into an eye of the patient at a third plurality of points in time during the phacoemulsification operation;
assessing the clinical index based on a following formula:
$$a {\int }_{t} {e}^{\vert IOP ( t ) - target \_ IOP )} dt + b {\int }_{t} E ( t ) dt + c {\int }_{t} B ( t ) dt$$
$$or$$
$$a {\int }_{t} {e}^{max ( 0 , IOP ( t ) - target \_ IOP )} dt + b {\int }_{t} E ( t ) dt + c {\int }_{t} B ( t ) dt$$
wherein
t is time from the beginning of the phacoemulsification operation,
IOP(t) is a value of the first sequence at time t,
target(IOP) is an IOP value desired to be maintained during the phacoemulsification operation,
E(t) is value of the second sequence at time t,
B(t) is value of the third sequence at time t, and
a, b, and c are predetermined numerical weights; and
providing to a user of the phacoemulsification system an indication of the clinical index.

20. A computer program product comprising a non-transitory computer readable medium retaining program instructions, which instructions when read by a processor, cause the processor to perform:
providing a medical system and at least one sensor coupled with the medical system for sensing a parameter within a body of the patient;
obtaining a first sequence of measured values, the first sequence of values indicating measurements taken by the at least one sensor at a plurality of points in time during the medical procedure;
assessing the clinical index based at least on a function of the first sequence of measured values, the function involving a deviation of each value of the first sequence of measured values from a threshold; and
providing to a user of the medical system an indication of the clinical index.
